(19) United States
(12) Patent Application Publication
(10) Pub. No.: US 2026/0335637 A1
(43) Pub. Date: Oct. 29, 2026

(54) SKIN CARE DEVICE

(71) Applicant: LG H&H Co., Ltd., Seoul (KR)

(72) Inventors: Sung Taek Oh, Seoul (KR); Sung Hwan Kim, Seoul (KR); Hyun Woo Kim, Seoul (KR)

(21) Appl. No.: 19/111,785

(22) PCT Filed: Jul. 28, 2023
(86) PCT No.: PCT/KR2023/011050

§ 371 Date: Mar. 13, 2025

(30) Foreign Application Priority Data
Sep. 13, 2022 (KR) 10-2022-0115076

(51) Int. Cl. A61N 1/32; A61N 1/06; A61N 1/08; A61N 1/40

(52) U.S. Cl. CPC A61N 1/328; A61N 1/06; A61N 1/08; A61N 1/403

(57) ABSTRACT

A skin care device according to an embodiment of the present disclosure includes a body including a first terminal; and a head portion connected to the body and detachable from the body, wherein the head portion includes: a housing including an accommodation space therein; a substrate disposed in the housing; an electrode portion disposed in the housing and disposed on the substrate; and a second terminal located below the substrate and electrically connected to the first terminal of the body; wherein the electrode portion includes: a first electrode disposed at a center of the head portion; a second electrode surrounding a periphery of the first electrode and disposed alternately at equal intervals; a third electrode surrounding a periphery of the second electrode and disposed alternately at equal intervals; and a fourth electrode surrounding a periphery of the third electrode and disposed alternately at equal intervals, wherein the first, second, third and fourth electrodes operate in a first RF mode, and wherein the first RF mode is applied to two of the first, second, third, and fourth electrodes at different frequencies alternately at a predetermined period, so that high-frequency thermal energy is evenly transmitted from an upper dermal layer to a lower dermal layer.

### TECHNICAL FIELD

The present invention relates to a skin care device that applies different frequencies to a plurality of electrodes alternately at a predetermined period so that high-frequency thermal energy is evenly transmitted from an upper dermal layer to a lower dermal layer, and forms a passage for movement of cosmetics (formulations) in electrodes disposed inside the plurality of electrodes and does not form a passage for movement in electrodes disposed at the outermost side, so that cosmetics are evenly spread only inside the head during use.

### BACKGROUND ART

Human skin may be contaminated by external activities, and wrinkles may be formed due to aging, hormonal changes, and the like. Recently, as interest in skin has increased, a cleansing device capable of effectively removing skin contamination, and various devices for skin beauty and anti-aging are being developed.

For example, a device for providing high-frequency, infrared energy to the skin is being developed. The device is a device that applies heat energy to the skin, and by applying heat energy to the skin, it is possible to improve the elasticity of the applied skin region, or to effectively provide a cosmetic product inside the skin.

In addition, a device for providing sound waves and light to the skin is being developed. For example, sonophoresis and laserporation devices capable of providing a cosmetic product to the inside of the skin using ultrasound or laser light are being developed. The devices may form a path for injection of cosmetics into the skin using ultrasound or laser light, thereby increasing the penetration rate of skin care products into the skin.

In addition, a device for physically providing a beauty product to the skin using a small current is being developed. For example, the devices are devices using iontophoresis, electroporation, electroosmosis, etc. It is a device that can be effectively provided inside the skin.

That is, various devices capable of providing skin beauty and anti-aging functions using light energy, sound waves, electric current, vibration, and the like are being developed.

In general, a cosmetic device using a high-frequency is a device that takes care of the skin by applying electrical stimulation to the skin, and may provide electrical stimulation in a high-frequency band to the skin. If a high-frequency device is described in detail, when high-frequency waves are applied to the skin, deep heat, which is frictional heat, may be generated as ions in the cell move from the cathode to the anode. The deep heat is about 40 °C to about 50 °C, and the skin may be sterilized by the deep heat. In addition, due to the deep heat, the blood vessels inside the skin can be expanded, the metabolic function can be smoothed, and there is an effect that the cosmetic product can be effectively provided inside the skin.

To this end, the device using the high-frequency includes an electrode formed on the surface, and the electrode may come into contact with the user's skin. However, when the device is used on a curved skin region such as a face, contact between the electrode and the skin may not be smooth. Accordingly, there is a problem in that the high-frequency is excessively provided to some areas, which may cause skin damage such as burns, and the high-frequency is not provided to some other areas.

In addition, the electrode of the device generally has a shape protruding from the surface of the device for contact with the skin. In this case, when the user grips the device and massages the skin, there is a problem in that it is difficult to naturally massage the curved skin regions such as both cheeks, chin, and both eyes by the protruding electrodes.

Accordingly, there is a need for a new skin care device capable of solving the above problems.

### DISCLOSURE

### Technical Problem

The present invention has been invented to solve the above-described problems of the prior art, and an object of the present invention is to provide a skin care device that allows high-frequency thermal energy to be evenly transmitted from a upper dermal layer to a lower dermal layer, and allows cosmetics to be evenly spread only inside the head during use.

### Technical Solution

A skin care device according to one aspect of the present invention may include a body including a first terminal; and a head portion connected to the body and detachable from the body, wherein the head portion includes: a housing including an accommodation space therein; a substrate disposed in the housing; an electrode portion disposed in the housing and disposed on the substrate; and a second terminal located below the substrate and electrically connected to the first terminal of the body; wherein the electrode portion includes: a first electrode disposed at a center of the head portion; a second electrode surrounding a periphery of the first electrode and disposed alternately at equal intervals; a third electrode surrounding a periphery of the second electrode and disposed alternately at equal intervals; and a fourth electrode surrounding a periphery of the third electrode and disposed alternately at equal intervals, wherein the first, second, third and fourth electrodes operate in a first RF mode, and wherein the first RF mode is applied to two of the first, second, third, and fourth electrodes at different frequencies alternately at a predetermined period, so that high-frequency thermal energy is evenly transmitted from an upper dermal layer to a lower dermal layer.

Specifically, the first RF mode may set frequencies to be used for the first, second, third, and fourth electrodes to four frequencies in a range of 1 MHz to 3 MHz (high-frequency), and the four frequencies may be set to a first frequency, a second frequency, a third frequency, and a fourth frequency in an order from longest to shortest frequency.

Specifically, the first frequency may be set to 2.2 MHz, the second frequency may be set to 1.8 MHz, the third frequency may be set to 1.4 MHz, and wherein the fourth frequency may be set to 1.0 MHz.

Specifically, the skin care device may further comprise an acceleration mode for rapid thermal transfer to a skin before operating the first RF mode, wherein one of acceleration level 1, acceleration level 2, and acceleration level 3 which are set in an order from longest to shortest time may be selected and operated in the acceleration mode, wherein the third and fourth electrodes may be supplied with a current of the first frequency at a duty of 100%, wherein a negative current may be applied to one of the third and fourth electrodes and a positive current may be applied to an other electrode.

Specifically, the first RF mode may be executed for a certain period of time from when the acceleration mode is completed and may be automatically stopped after the certain period of time lapses, and the first RF mode may be divided into step 1 and step 2 which are separately executed in a manner of which when two of the first, second, third, and fourth electrodes are alternately applied with two of the frequency currents of the first, two, three, and four frequencies at a predetermined period, no current is supplied to the other two electrodes.

Specifically, the first RF mode may be configured to apply the currents of the second frequency and the fourth frequency alternately to each of the first and second electrodes several times to several tens of times at a period of less than 1 second during a first set time in the step 1, and may apply the currents of the first frequency and the third frequency alternately to the third and fourth electrodes several times to several tens of times at the period of less than 1 second during the first set time in the step 2, and wherein a negative current may be applied to one of the two first and second electrodes or the two third and fourth electrodes, and a positive current may be applied to an other electrode, and the step 1 and the step 2 may alternately be executed with a gap of a second set time during which no current is supplied to each of the first, second, third, and fourth electrodes.

Specifically, the first set time may become longer from the acceleration level 1 to the acceleration level 3, and the second set time may become relatively shorter from the acceleration level 1 to the acceleration level 3.

Specifically, the first RF mode may be repeatedly executed for the certain period of time in an order of the step 1->the step 2->the step 1->the step 2 after the acceleration mode.

Specifically, the first, second, third, and fourth electrodes may be operated in a first EMS mode instead of the first RF mode, the first EMS mode may be divided into step 3, step 4, and step 5 which are separately executed in a manner of which when a current of a fifth frequency is applied to two of the first, second, third, and fourth electrodes at a predetermined period, no current is supplied to other two electrodes, wherein the first EMS mode may be configured to apply the current of the fifth frequency to each of the third and fourth electrodes in the step 3, may apply the current of the fifth frequency to each of the first and fourth electrodes in the step 4, and may apply the current of the fifth frequency to each of the first and second electrodes in the step 5, and wherein a negative current may be applied to one of the two third and fourth electrodes, the two first and fourth electrodes, or the two first and second electrodes, and a positive current may be applied to an other electrode.

Specifically, in the first EMS mode, one of stimulation level 1, stimulation level 2, and stimulation level 3 which are set in an order from shortest to longest may be selected and executed so that a stimulation time in each of the step 3, the step 4, and the step 5 is different, and between each of the step 3, the step 4, and the step 5, no current may be supplied to each of the first, second, third, and fourth electrodes for a certain period of time.

Specifically, the first EMS mode may be repeatedly executed for a certain period of time in an order of the step 3->step 4->step 5->step 4->step 3->step 4->step 5->step 4.

Specifically, the first, second, third, and fourth electrodes may be operated in a RF+EMS mode instead of the first RF mode, the RF+EMS mode may be divided into step 6, step 7, and step 8 which are separately executed in a manner of which when the second RF mode is operated in two of the first, second, third, and fourth electrodes, and the second EMS mode may be operated in other two electrodes.

Specifically, the first and second electrodes may be executed in the second RF mode and the third and fourth electrodes may be executed in the second EMS mode in the step 6, the second and third electrodes may be executed in the second RF mode and the first and fourth electrodes may be executed in the second EMS mode in the step 7, and the third and fourth electrodes may be executed in the second RF mode and the first and second electrodes may be executed in the second EMS mode in the step 8.

Specifically, the second RF mode may set frequencies to be used for the first, second, third, and fourth electrodes to four frequencies in a range of 1 MHz to 3 MHz (high frequency), wherein the four frequencies may be set to a first frequency, a second frequency, a third frequency, and a fourth frequency in an order from longest to shortest frequency, and wherein the second EMS may set frequencies to be used for the first, second, third, and fourth electrodes to five frequencies in a low frequency range.

Specifically, the skin care device may further comprise an acceleration mode for rapid thermal transfer to a skin before operating the second RF mode, wherein one of acceleration level 1, acceleration level 2, and acceleration level 3 which are set in an order from longest to shortest time may be selected and operated in the acceleration mode, wherein the third and fourth electrodes may be supplied with a current of the first frequency at a duty of 100%, wherein a negative current may be applied to one of the third and fourth electrodes and a positive current is applied to an other electrode.

Specifically, second RF mode may be configured to apply the currents of the first frequency and the second frequency alternately to each of the first and second electrodes several times to several tens of times at a period of less than 1 second during a first set time in the step 6, may apply the currents of the second frequency and the third frequency alternately to each of the second and third electrodes several times to several tens of times at the period of less than 1 second during the first set time in the step 7, and may apply the currents of the third frequency and the fourth frequency alternately to the third and fourth electrodes several times to several tens of times at the period of less than 1 second during the first set time in the step 8, wherein a negative current may be applied to one electrode of the two first and second electrodes, the two second and third electrodes, or the two third and fourth electrodes, and a positive current may be applied to an other electrode, wherein the step 6, the step 7, and the step 8 may alternately be executed with a gap of a second set time during which no current is supplied to each of the first, second, third, and fourth electrodes, wherein the first set time may become longer from the acceleration level 1 to the acceleration level 3, wherein the second set time may become relatively shorter from the acceleration level 1 to the acceleration level 3, wherein the second EMS mode may be configured to apply a current of the fifth frequency to each of the third and fourth electrodes in the step 6, may apply the current of the fifth frequency to each of the first and fourth electrodes in the step 7, and may apply the current of the fifth frequency to each of the first and second electrodes in the step 8, and wherein a negative current may be applied to one of the two third and fourth electrodes, the two first and fourth electrodes, or the two first and second electrodes, and a positive current may be applied to an other electrode.

Specifically, in the second EMS mode, one of stimulation level 1, stimulation level 2, and stimulation level 3 which are set in an order from shortest to longest may be selected and executed so that a stimulus time in each of the step 6, the step 7, and the step 8 is different, and between each of the step 6, the step 7, and the step 8, no current may be supplied to each of the first, second, third, and fourth electrodes for a certain period of time.

Specifically, the RF+EMS mode may be repeatedly executed for a certain period of time in an order of the step 6->step 7->step 8->step 7->step 6->step 7->step 8->step 7.

### Advantageous Effects

A skin care device according to the present invention, by alternately applying different frequencies to a plurality of electrodes at a predetermined period, high-frequency thermal energy may be evenly transmitted from an upper dermal layer to a lower dermal layer in a RF mode, thereby maximizing the skin improvement effect.

In addition, the skin care device according to the present invention may form a movement passage for cosmetics (formulations) in electrodes disposed on the inside of a plurality of electrodes, so that cosmetics may be evenly spread to the electrodes along the movement passage during use.

In addition, the skin care device according to the present invention does not form a movement passage in the outermost electrode of the plurality of electrodes, so that cosmetics may be evenly spread only inside the head during use.

In addition, the skin care device according to the present invention uses a combination of high-frequency and low-frequency electrodes, so that high-frequency thermal energy may be evenly transmitted from the upper dermal layer to the lower dermal layer by the electrode operating in RF mode, and muscle pain may be eliminated and muscles may be activated by the electrode operating in EMS (Electrical Muscle Stimulation) mode, thereby further maximizing the skin improvement effect.

In addition, the skin care device according to the present invention may provide various wavelength bands of light to the user's skin through a light emitting device, thereby maximizing the cosmetic effect such as inducing the formation of collagen on the user's surface and reducing fine wrinkles, as well as providing the therapeutic effect such as calming the area to which light of the various wavelength bands is applied or alleviating the formed inflammation.

In addition, the skin care device according to the present invention enables a head portion provided with the plurality of electrodes to be detached from the body, so that only the head portion may be replaced when the head portion is damaged or broken, so that high efficiency may be achieved, and when the user wants to use an additional function other than the function included in the head portion, the head portion including the additional function may be connected to the body without the need to separately purchase or include the skin care device for the additional function, so that the user may use the skin care device economically and efficiently.

In addition, the skin care device according to the present invention may supply at least one current selected from a galvanic current and a microcurrent as well as a high-frequency/low-frequency current to the plurality of electrodes, thereby forming a deep heat in the user's skin, activating muscles, promoting the production of adenosine triphosphate (ATP), and effectively providing cosmetics or drugs into the skin.

In addition, the skin care device according to the present invention, the surface of the head portion where the plurality of electrodes are in contact with the user's skin has a curved shape, so that the plurality of electrodes may effectively adhere to a relatively curved area such as the chin, both cheeks, and the eye, and the plurality of electrodes effectively come into contact with the skin without being separated from the skin, so that the skin of the user may be effectively massaged.

### DESCRIPTION OF DRAWINGS

FIG. 1 is a perspective view of a skin care device according to an embodiment of the present invention.

FIG. 2 is an exploded perspective view of the skin care device according to an embodiment of the present invention.

FIG. 3 is a rear perspective view of a head portion in the skin care device according to an embodiment of the present invention.

FIG. 4 is a front view of the head portion in the skin care device according to an embodiment of the present invention.

FIG. 5 is a cross-sectional view of the head portion taken along line A-A′ of FIG. 4.

FIG. 6 is an exploded perspective view of the head portion in the skin care device according to an embodiment of the present invention.

FIG. 7 is a first perspective view of an electrode portion in the skin care device according to an embodiment of the present invention.

FIG. 8 is a second perspective view of the electrode portion in the skin care device according to an embodiment of the present invention.

FIG. 9 is an exploded perspective view of the electrode portion in the skin care device according to an embodiment of the present invention.

FIG. 10 is a front view of a cover of a housing in the skin care device according to an embodiment of the present invention.

FIGS. 11(a) and 11(b) are diagrams comparing the transfer of thermal energy to the dermal layer in the skin care device of the present invention and a skin care device according to the comparative invention.

### MODES OF THE INVENTION

The objects, particular advantages, and novel features of the invention will become more apparent from the following detailed description and the preferred embodiments, which are in connection with the accompanying drawings. In the specification, it should be noted that reference numerals are added to the components in each drawing, so that the same components have the same numerals as much as possible, even if they are shown in other drawings. In addition, in describing the present invention, if it is determined that a specific description of the related known art may unnecessarily obscure the gist of the present invention, a detailed description thereof will be omitted.

Hereinafter, preferred embodiments of the present invention will be described in detail with reference to the accompanying drawings.

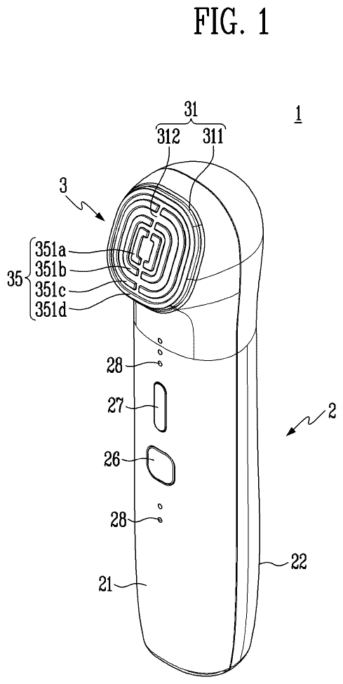

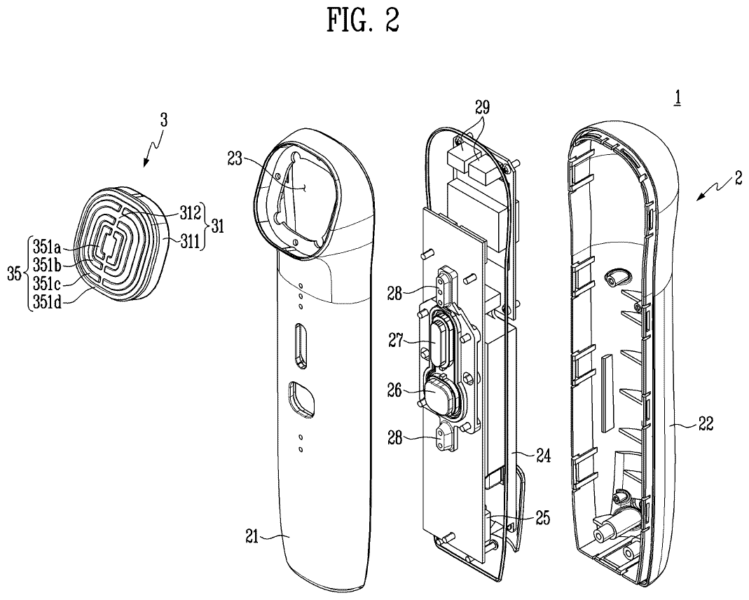

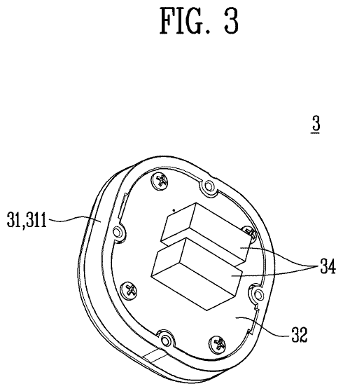

FIG. 1 is a perspective view of a skin care device according to an embodiment of the present invention. FIG. 2 is an exploded perspective view of the skin care device and FIG. 3 is a rear perspective view of a head portion in the skin care device.

Referring to FIGS. 1 to 3, a skin care device 1 according to an embodiment of the present invention may include a body 2 and a head portion 3.

The body 2 may have a shape that is easy for a user to grasp by hand, and may be connected to the head portion 3. The body 2 may have a shape extending in one direction. For example, the body 2 may have a shape extending in a vertical direction. The body 2 may have a bar or stick shape that extends in one direction, such as a cylinder or a polygonal column. Accordingly, the user may control the position of the skin care device 1 by grasping the body 2 by hand.

The body 2 may include a front cover 21, a rear cover 22, and a recess 23. The body 2 may have an accommodation space inside by the front cover 21 and the rear cover 22. Various devices for operating the skin care device 1 such as a battery 24, a charging terminal 25, a switch 26, a function button 27, an indicator lamp 28, and a first terminal 29 may be disposed in the accommodation space of the body 2.

The recess 23 may be located on one side of an upper portion of the front cover 21 of the body 2. The recess 23 may have a concave shape in a direction toward inside of the body 2 from one side of the front cover 21 of the body 2, and may be configured to allow the head portion 3 to be detachable.

The battery 24 may supply power to the skin care device 1. For example, the battery 24 may supply power to each of the body 2 and the head portion 3 connected to the body 2. The battery 24 may include one selected from primary batteries such as manganese (Mn) batteries, alkaline batteries, mercury batteries, silver oxide batteries, or the like. In addition, the battery 24 may include one selected from secondary batteries such as nickel-cadmium (Ni—Cd) batteries, nickel-hydrogen (Ni-MH) batteries, and lithium-ion (Li-ion) batteries.

When the skin care device 1 includes a rechargeable battery 24, a cover for detaching and attaching the battery 24 may be disposed on the rear cover 22 of the body 2. Alternatively, when detaching and attaching is difficult as the battery 24 is disposed inside the body 2, the charging terminal 25 may be located on the rear cover 22 of the skin care device 1. The charging terminal 25 is electrically connected to the battery 24 disposed inside the body 2, and a portion of the charging terminal 25 may be exposed outside the rear cover 22 of the body 2. The user may charge the battery 24 by connecting a USB port or cable for charging the battery 24 to the exposed charging terminal 25. Alternatively, the skin care device 1 may be provided so as to be wirelessly rechargeable. More specifically, the skin care device 1 may be wirelessly rechargeable by a coil or the like disposed in the body 2. For example, the skin care device 1 may charge the battery 24 by a wireless charging method such as a magnetic resonance method or a magnetic induction method.

The switch 26 may be installed so as to partially protrude to the outside of the front cover 21 of the body 2, and the installation location is not limited, and the switch 26 may be installed at any location as long as conveniently operated by the user. The switch 26 may be a physical switch including an elastic body such as a spring. In addition, the switch 26 may be an electrical switch in which a touch electrode or the like is formed. The user may turn on/off the operation of the skin care device 1 through the switch 26.

The function button 27 may be installed so as to partially protrude to the outside of the front cover 21 of the body 2, and the installation location is not limited, and the function button 27 may be installed at any location as long as conveniently operated by the user. The function button 27 may select a function of the skin care device 1. For example, the function button 27 may select a function such as a first RF mode, a first EMS mode, an RF+EMS mode, a light emission mode, or the like.

The indicator lamp 28 may be installed so as to be partially exposed outside the front cover 21 of the body 2. For example, the indicator lamp 28 may be disposed adjacent to the switch 26 and/or the functional button 27, and may display the current state of the skin care device 1, such as the switching state of the skincare device 1 or the functional state of the first RF mode, the first EMS mode, the light emitting mode, or the like.

The first terminal 29 may be electrically connected to the battery 24. The first terminal 29 may be electrically connected to a second terminal 34 of the head portion 3 coupled through the recess 23.

The head portion 3 may be connected to the body 2, and the head portion 3 may be physically connected with the body 2. For example, the head portion 3 may have a shape corresponding to the recess 23 located in the front cover 21 of the body 2, and may be inserted into and connected to the recess 23 at a predetermined depth. The head portion 3 may be detachable from the body 2.

In addition, the head portion 3 may be electrically connected to the body 2. For example, the second terminal 34 is located on a lower surface of the head portion 3, and the head portion 3 may be electrically connected through the first terminal 29 located inside the accommodation space of the body 2. Accordingly, the battery 24 disposed in the body 2 may supply power to the head portion 3.

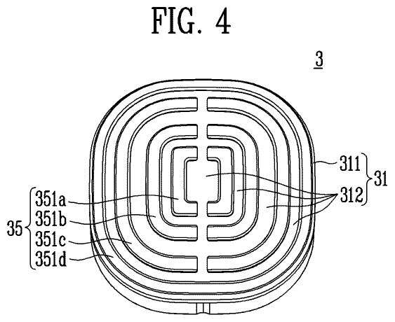

Hereinafter, the head portion 3 will be described in detail with reference to FIGS. 4 to 10.

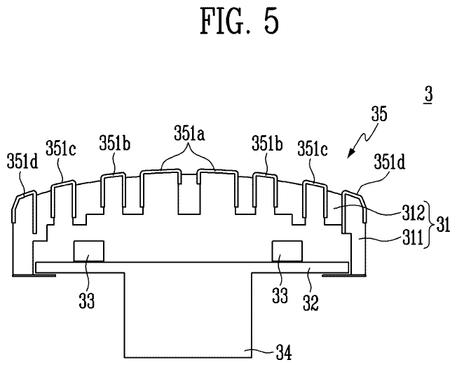

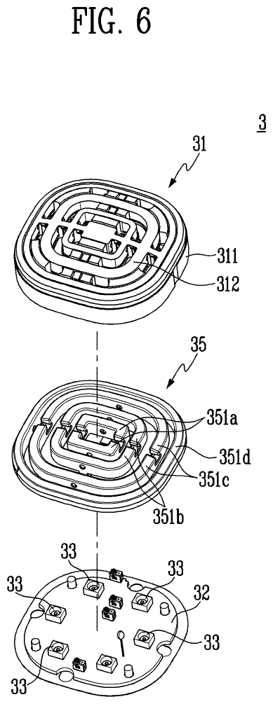

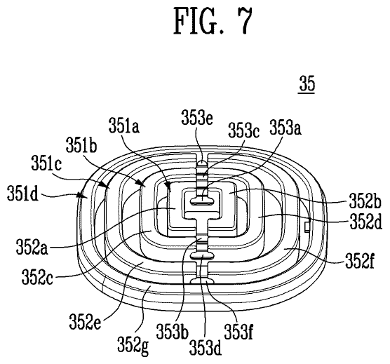

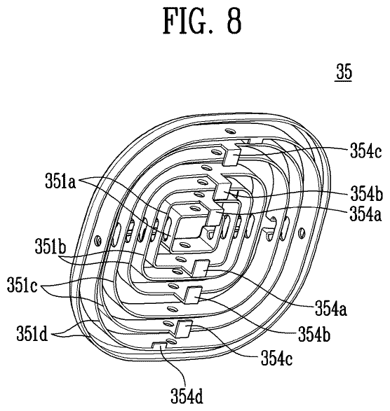

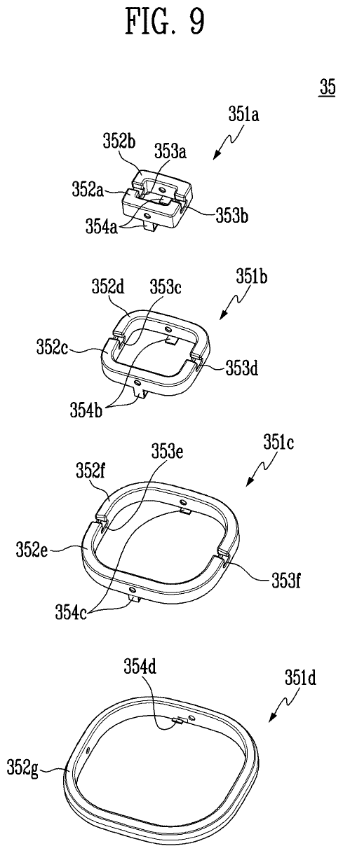

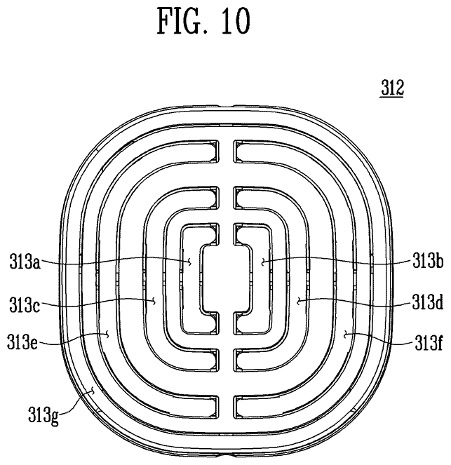

FIG. 4 is a front view of the head portion in the skin care device according to an embodiment of the present invention, FIG. 5 is a cross-sectional view of the head portion taken along line A-A′ of FIG. 4; FIG. 6 is an exploded perspective view of the head portion in the skin care device according an embodiment of the present invention, FIG. 7 is a first perspective view of an electrode portion in the skin care device according to the embodiment of the invention, FIG. 8 is a second perspective view of the electrode portion in the skin care device according to an embodiment of the present invention, FIG. 9 is an exploded perspective view of the electrode portion in the skin care device according to an embodiment of the present invention, and FIG. 10 is a front view of a cover of a housing in the skin care device according to an embodiment of the present invention.

The head portion 3 may include a housing 31, a substrate 32, a light-emitting element 33, the second terminal 34, and an electrode portion 35.

The housing 31 may include an edge 311 and a cover 312 covering an upper portion of the edge 311, such that a lower portion is open and has an accommodation space therein. The substrate 32, the light-emitting element 33, the second terminal 34, and the electrode portion 35 may be accommodated in the accommodation space of the housing 31. The edge 311 may have a size that fixes edges of the substrate 32 and the electrode portion 35.

The cover 312 may be located on the upper portion of the edge 311. The cover 312 may be translucent or opaque, and may include a material capable of transmitting light emitted from the light emitting device 33. In addition, the cover 312 may include a material that is light and may prevent damage from external impact, contact, or the like. For example, the cover 312 may include at least one of a resin, a ceramic, a metal, or the like, and may transmit light emitted from the light-emitting element 33 and may have improved reliability to the external environment.

The cover 312 may have a structure that is capable of detaching and attaching to the edge 311. However, the embodiment is not limited thereto, and the cover 312 and the edge 311 may also be integrally formed.

As described above, the housing 31 including the edge 311 and the cover 312 may include a plurality of holes 313a, 313b, 313c, 313d, 313e, 313f, and 313g so that a portion thereof may be exposed to the outside when the electrode portion 35 is accommodated. The plurality of holes 313a, 313b, 313c, 313d, 313e, 313f, and 313g will be described later together with the electrode portion 35 in consideration of the fact that they are related to each other.

The substrate 32 may be accommodated in the housing 31. A plurality of light-emitting elements 33 may be disposed on the upper portion of the substrate 32, and the second terminal 34 electrically connected to the first terminal 29 of the body 2 may be located below the substrate 32.

The plurality of light-emitting elements 33 may be electrically connected to the substrate 32. The plurality of light-emitting elements 33 may be disposed on an upper surface of the substrate 32. The plurality of light-emitting elements 33 may be disposed in an edge region of the substrate 32.

The plurality of light-emitting elements 33 may emit light in the visible light band when the light-emitting mode is selected by the function button 27. More specifically, the plurality of light-emitting elements 33 may be visible light-emitting elements, and may emit light having a wavelength of about 400 nm or more. For example, the plurality of light-emitting elements 33 may emit at least one of green light in a wavelength band of about 450 nm to about 580 nm, orange light in a wavelength band of about 590 nm to about 620 nm, and red light in a wavelength band of about 620 nm to about 750 nm.

In an embodiment, the light-emitting mode may be used alone, in the first RF mode, the first EMS mode, or the RF+EMS mode. The skin care device 1 of the present invention may operate the light emitting mode together with the first RF mode, the first EMS mode, or the RF+EMS mode.

The electrode portion 35 may be accommodated inside the housing 31. The electrode portion 35 may be disposed on the substrate 32. The electrode portion 35 may include a material that is harmless to the human body. More specifically, the electrode portion 35 may not be deformed by contact pressure with the skin and may include a conductive metal material. For example, the electrode portion 35 may include at least one metal among aluminum (Al), copper (Cu), zinc (Zn), iron (Fe), nickel (Ni), chromium (Cr), silver (Ag), gold (Au), platinum (Pt), stainless steel (SUS), and alloys thereof.

The electrode portion 35 may be electrically connected to the substrate 32 and may be in contact with the user's skin. The electrode portion 35 may provide at least one of a galvanic current and a microcurrent as well as a high-frequency/low-frequency current to the user's skin.

The electrode portion 35 may include a plurality of electrodes 351a, 351b, 351c, and 351d. For example, the electrode portion 35 may include the first electrode 351a, the second electrode 351b, the third electrode 351c, and the fourth electrode 351d that are spaced apart from each other.

The first electrode 351a may be disposed at a center of the head portion 3, the second electrode 351b may be disposed at equal intervals surrounding a periphery of the first electrode 351b, the third electrode 351c may be disposed at equal intervals surrounding a periphery of the second electrode 352b, and the fourth electrode 351d may be disposed at equal intervals surrounding a periphery of the third electrode 352c.

The first electrode 351a may have a half square ring shape in a planar shape, and is not limited thereto, and may have various shapes such as an annular shape and a donut shape. The first electrode 351a may have a first width and a first height, and may have a U-shape in which a cross-sectional shape is reversed. Accordingly, the weight of the first electrode 351a may be reduced, and furthermore, the total weight and manufacturing cost of the head portion 3 may be reduced.

The first electrode 351a may include a first contact portion 352a and a second contact portion 352b exposed to the outside of the head portion 3. The first contact portion 352a and the second contact portion 352b may be electrodes in contact with the user's skin.

The first contact portion 352a and the second contact portion 352b may have shapes corresponding to each other. For example, the first contact portion 352a and the second contact portion 352b may have a U-shape in which the cross-sectional shape is reversed.

The first contact portion 352a and the second contact portion 352b may face each other in a first direction (horizontal direction) and may be spaced apart from each other. In addition, the first contact portion 352a and the second contact portion 352b may be symmetrical with respect to each other based on a virtual line in a second direction (vertical direction).

The first contact portion 352a and the second contact portion 352b may include an upper surface facing and in contact with the user's skin. The upper surface of each of the first contact portion 352a and the second contact portion 352b may be disposed above an upper surface of the cover 312 of the housing 31. Accordingly, the first contact portion 352a and the second contact portion 352b may be in contact with the user's skin and may provide at least one of a galvanic current and a microcurrent as well as a high-frequency/low-frequency current to the skin.

The first electrode 351a may further include a first connection portion 353a and a second connection portion 353b. The first connection portion 353a and the second connection portion 353b may be disposed at both ends of the first contact portion 352a and the second contact portion 352b, respectively, to connect the first contact portion 352a and the second contact portion 352b.

The first connection portion 353a and the second connection portion 353b may have shapes corresponding to each other. For example, the first connection portion 353a and the second connection portion 353b may face each other in the second direction (vertical direction) and may be spaced apart from each other. In addition, the first connection portion 353a and the second connection portion 353b may be symmetrical with respect to a virtual line in the first direction (horizontal direction).

The first connection portion 353a and the second connection portion 353b may not be exposed to the outside of the head portion 3. For example, the first connection portion 353a and the second connection portion 353b may have a shape extending by being bent toward the substrate 32 at the both ends of the first contact portion 352a and the second contact portion 352b, respectively.

The first connection portion 353a and the second connection portion 353b may have a lower height than the first contact portion 352a and the second contact portion 352b. An upper surface of each of the first connection portion 353a and the second connection portion 353b may be disposed lower than the upper surface of the cover 312. The first connection portion 353a and the second connection portion 353b may have a concave shape at the both ends of the first contact portion 352a and the second contact portion 352b, respectively.

The first connection portion 353a and the second connection portion 353b may provide a passage for movement of cosmetics (formulations), so that the cosmetics may be evenly spread to the first electrode 351a along the first connection portion 353a and the second connection portion 353b when used.

In addition, the embodiment may include a first contact portion 354a connecting the first electrode 351a and the substrate 32. The first contact portion 354a may extend in a direction from at least one of the first contact portion 352a, the second contact portion 352b, the first connection portion 353a, and the second connection portion 353b toward the substrate 32 and may allow a set current to be applied to the first electrode 351a.

The second electrode 351b may have a shape corresponding to the first electrode 351a. For example, the second electrode 351b may have a half square ring shape in a planar shape, and is not limited thereto, and may have various shapes such as an annular shape and a donut shape. The second electrode 351b may have a second width and a second height, and may have a U-shape in which a cross-sectional shape is reversed. Accordingly, the weight of the second electrode 351b may be reduced, and furthermore, the total weight and manufacturing cost of the head portion 3 may be reduced.

The second electrode 351b may include a third contact portion 352c and a fourth contact portion 352d exposed to the outside of the head portion 3. The third contact portion 352c and the fourth contact portion 352d may be electrodes in contact with the user's skin.

The third contact portion 352c and the fourth contact portion 352d may have shapes corresponding to each other. For example, the third contact portion 352c and the fourth contact portion 352d may have a U-shape in which the cross-sectional shape is reversed.

The third contact portion 352c and the fourth contact portion 352d may face each other in the first direction (horizontal direction) and may be spaced apart from each other. In addition, the third contact portion 352c and the fourth contact portion 352d may be symmetrical with respect to each other based on the virtual line in the second direction (vertical direction).

The third contact portion 352c and the fourth contact portion 352d may include an upper surface facing and in contact with the user's skin. The upper surface of each of the third contact portion 352c and the fourth contact portion 352d may be disposed above the upper surface of the cover 312 of the housing 31. Accordingly, the third contact portion 352c and the fourth contact portion 352d may be in contact with the user's skin and may provide at least one of a galvanic current and a microcurrent as well as a high-frequency/low-frequency current to the skin.

The second electrode 351b may further include a third connection portion 353c and a fourth connection portion 353d. The third connection portion 353c and the fourth connection portion 353d may be disposed at both ends of the third contact portion 352c and the fourth contact portion 352d, respectively, to connect the third contact portion 552c to the fourth contact portion 552d.

The third connection portion 353c and the fourth connection portion 353d may have shapes corresponding to each other. For example, the third connection portion 353c and the fourth connection portion 353d may face each other in the second direction (vertical direction) and may be spaced apart from each other. In addition, the third connection portion 353c and the fourth connection portion 353d may be symmetrical with respect to the virtual line in the first direction (horizontal direction).

The third connection portion 353c and the fourth connection portion 353d may not be exposed to the outside of the head portion 3. For example, the third connection portion 353c and the fourth connection portion 353d may have a shape extending by being bent toward the substrate 32 at the both ends of the third contact portion 352c and the fourth contact portion 352d, respectively.

The third connection portion 353c and the fourth connection portion 353d may have a lower height than the third contact portion 352c and the fourth contact portion 352d. An upper surface of each of the third connection portion 353c and the fourth connection portion 353d may be disposed below an upper surface of the cover 312. The third connection portion 353c and the fourth connection portion 353d may have a concave shape at the both ends of the third contact portion 352c and the fourth contact portion 352d, respectively.

The third connection portion 353c and the fourth connection portion 353d may provide a passage for movement of the cosmetics (formulations), so that the cosmetic product may be evenly spread to the second electrode 351b along the third connection portion 353c and the fourth connecting portion 353d when used.

In addition, the embodiment may include a second contact portion 354b connecting the second electrode 351b and the substrate 32. The second contact portion 354b may extend in a direction from at least one of the third contact portion 352c, the fourth contact portion 352d, the third connection portion 353c, and the fourth connection portion 353d toward the substrate 32 and may allow a set current to be applied to the second electrode 351b.

The third electrode 351c may have a shape corresponding to the second electrode 351b. For example, the third electrode 351c may have a half square ring shape in a planar shape, and is not limited thereto, and may have various shapes such as an annular shape and a donut shape. The third electrode 351c may have a third width and a third height, and may have a U-shape in which a cross-sectional shape is reversed. Accordingly, the weight of the third electrode 351c may be reduced, and furthermore, the total weight and manufacturing cost of the head portion 3 may be reduced.

The third electrode 351c may include a fifth contact portion 352e and a sixth contact portion 352f exposed to the outside of the head portion 3. The fifth contact portion 352e and the sixth contact portion 352f may be electrodes in contact with the user's skin.

The fifth contact portion 352e and the sixth contact portion 352f may have shapes corresponding to each other. For example, the fifth contact portion 352e and the sixth contact portion 352f may have a U-shape in which a cross-sectional shape is reversed.

The fifth contact portion 352e and the sixth contact portion 352f may face each other in the first direction (horizontal direction) and may be spaced apart from each other. In addition, the fifth contact portion 352e and the sixth contact portion 352f may be symmetrical with respect to each other based on the virtual line in the second direction (vertical direction).

The fifth contact portion 352e and the sixth contact portion 352f may include an upper surface facing and in contact with the user's skin. The upper surface of each of the fifth contact portion 352e and the sixth contact portion 352f may be disposed above the upper surface of the cover 312 of the housing 31. Accordingly, the fifth contact portion 352e and the sixth contact portion 352f may be in contact with the user's skin and may provide at least one of a galvanic current and a microcurrent as well as a high-frequency/low-frequency current to the skin.

The third electrode 351c may further include a fifth connection portion 353e and a sixth connection portion 353f. The fifth connection portion 353e and the sixth connection portion 353f may be disposed at both ends of the fifth contact portion 352e and the sixth contact portion 352f, respectively, to connect the fifth contact portion 552e and the six contact portions 352f.

The fifth connection portion 353e and the sixth connection portion 353f may have shapes corresponding to each other. For example, the fifth connection portion 353e and the sixth connection portion 353f may face each other in the second direction (vertical direction) and be spaced apart from each other. In addition, the fifth connection portion 353e and the sixth connection portion 353f may be symmetrical with respect to the virtual line in the first direction (horizontal direction).

The fifth connection portion 353e and the sixth connection portion 353f may not be exposed to the outside of the head portion 3. For example, the fifth connection portion 353e and the sixth connection portion 353f may have a shape extending by being bent toward the substrate 32 at the both ends of the fifth contact portion 352e and the sixth contact portion 352f, respectively.

The fifth connection portion 353e and the sixth connection portion 353f may have a lower height than the fifth contact portion 352e and the sixth contact portion 352f. An upper surface of each of the fifth connection portion 353e and the sixth connection portion 353f may be disposed below the upper surface of the cover 312. The fifth connection portion 353e and the sixth connection portion 353f may have a concave shape at the both ends of the fifth contact portion 352e and the sixth contact portion 352f, respectively.

The fifth connection portion 353e and the sixth connection portion 353f may provide a passage for movement of cosmetics (formulations), so that the cosmetics may be evenly spread to the third electrode 351c along the fifth connection portion 353e and the sixth connecting portion 353f when used.

In addition, the embodiment may include a third contact portion 354c connecting the third electrode 351c and the substrate 32. The third contact portion 354c may extend in a direction from at least one of the fifth contact portion 352e, the sixth contact portion 352f, the fifth connection portion 353e, and the sixth connection portion 353f toward the substrate 32 and may allow a set current to be applied to the third electrode 351c.

The fourth electrode 351d may have a shape corresponding to the third electrode 351c. For example, the fourth electrode 351d may have a half square ring shape in a planar shape, but is not limited thereto, and may have various shapes such as an annular shape and a donut shape. The fourth electrode 351d may have a fourth width and a fourth height, and may have a U-shape in which a cross-sectional shape is reversed. Accordingly, the weight of the fourth electrode 351d may be reduced, and furthermore, the total weight and manufacturing cost of the head portion 3 may be reduced.

In this embodiment, the fourth electrode 351d may include a common seventh contact portion 352g in which the connection portions 353a, 353b, 353c, 353d, 353e, and 353f are omitted, unlike the first, second, and third electrodes 351a, 351b, and 351c described above.

The fourth electrode 351d may include the seventh contact portion 352g exposed to the outside of the head portion 3. The seventh contact portion 352g may be an electrode in contact with the user's skin.

The seventh contact portion 352g may have a U-shape in which the cross-sectional shape is reversed.

The seventh contact portion 352g may include an upper surface facing and in contact with the user's skin. The upper surface of the seventh contact portion 352g may be disposed above the upper surface of the cover 312 of the housing 31. Accordingly, the seventh contact portion 352g may be in contact with the user's skin and may provide at least one of a galvanic current and a microcurrent as well as a high-frequency/low-frequency current to the skin.

In this way, unlike the first, second, and third electrodes 351a, 351b, and 351c described above, the fourth electrode 351d of the present embodiment may not form the connecting portions 353a, 353b, 353c, 353d, 353e, and 353f that provide the movement path of the cosmetics, so that the cosmetics may be evenly spread only inside the head portion 3 when used.

In addition, the embodiment may include a fourth contact portion 354e connecting the fourth electrode 351d and the substrate 32. The fourth contact portion 354e may extend in a direction toward the substrate 32 from the seventh contact portion 352g, and a set current may be applied to the fourth electrode 351d.

In an embodiment, the first width of the first electrode 351a, the second width of the second electrode 351b, the third width of the third electrode 351c, and the fourth width of the fourth electrode 351d may each be the same or similar.

However, in an embodiment, the first height of the first electrode 351a, the second height of the second electrode 351b, the third height of the third electrode 351c, and the fourth height of the fourth electrode 351d may each be the same or similar, but it may be preferable to form them differently for the following reasons.

For example, the first height of the first electrode 351a disposed at the center of the head portion 3 may be the highest, and the fourth height of the fourth electrode 351d disposed at an outermost edge of the head portion may be the lowest, based on a horizontal plane.

For example, the height difference between adjacent electrodes in the first, second, third, and fourth electrodes 351a, 351b, 351c, and 351d may range from 5% to 25%. Preferably, the height difference between the adjacent electrodes in the first, second, third, and fourth electrodes 351a, 351b, 351c, and 351d may range from 5% to 20%. More preferably, the height difference between the adjacent electrodes in the first, second, third, and fourth electrodes 351a, 351b, 351c, and 351d may range from 5% to 15%.

When the height difference between the adjacent electrodes in the first, second, third, and fourth electrodes 351a, 351b, 351c, and 351d is greater than about 25% or less than about 5%, the contact characteristics of the user's skin and the contact portions 352a, 352b, 352c, 352d, 352e, 352f, and 352g may be degraded. For example, the user's skin area is divided into a flat skin area and curved skin areas such as both cheeks, chin, and eyes, and when the height difference between the adjacent electrodes in the first, second, third, and fourth electrodes 351a, 351b, 351c, and 351d exceeds about 25%, the contact characteristics of the contact portions 352a, 352b, 352c, 352d, 352e, 352f, and 352g in the flat skin area were degraded, and when the difference is less than about 5%, the contact characteristics in the curved skin area of the contact portions 352a, 352b, 352c, 352d, 352e, 352f, and 352g were degraded. In addition, when the height difference between the adjacent electrodes in the first, second, third, and fourth electrodes 351a, 351b, 351c, and 351d exceeds about 25%, a phenomenon in which cosmetics or drugs disposed between the head portion 3 and the user's skin move to an edge region of the head portion 3 or to the center region of the head portions 3 occurs.

As described above, the housing 31 including the edge 311 and the cover 312 may include the plurality of holes 313a, 313b, 313c, 313d, 313e, 313f, and 313g so that a portion thereof may be exposed to the outside when the electrode portion 35 is accommodated.

The cover 312 may include the first and second holes 313a and 313b formed in a region respectively corresponding to the first and second contact portions 352a and 352b of the first electrode 351a, the third and fourth holes 313c and 313d formed in a region respectively corresponding to the third and fourth contact portions 352c and 352d of the second electrode 351b, the fifth and sixth holes 313e and 313f formed in a region respectively corresponding to the fifth and sixth contact portions 352e and 352f of the third electrode 351c, and the seventh hole 313g formed in a region corresponding to the seventh contact portion 352g of the fourth electrode 351d.

More specifically, each of the first and second holes 313a and 313b may be disposed in a region vertically overlapping the first and second contact portions 352a and 352b, and may have a shape corresponding to the first and second contact portions 352a and 352b, respectively. A portion of the first electrode 351a, for example, a portion of the first contact portion 352a, may be inserted and disposed in the first hole 313a, and a portion of the second electrode 351a, for example, the portion of the first contact portion 352a, may be inserted and disposed in the second hole 313b.

In this case, an area of each of the first and second contact portions 352a and 352b is disposed below the cover 312, and the remaining area of each of first and second contact portions 352a and 352b may protrude from the upper surface of the cover 312 to be in contact with the user's skin.

In addition, each of the third and fourth holes 313c and 313d may be disposed in a region overlapping the third and fourth contact portions 352c and 352d in the vertical direction, and may have a shape corresponding to the third and fourth contact portions 352c and 352d, respectively. A portion of the second electrode 351b, for example, a portion of the third contact portion 352c, may be inserted and disposed in the third hole 313c, and a portion of the second electrode 351b, for example, a portion of the fourth contact portion 352d, may be inserted and disposed in the fourth hole 313d.

In this case, an area of each of the third and fourth contact portions 352c and 352d is disposed below the cover 312, and the remaining area of each of the fourth and third contact portions 352d and 352d may protrude from the upper surface of the cover 312 to be in contact with the user's skin.

In addition, each of the fifth and sixth holes 313e and 313f may be disposed in a region overlapping the fifth and sixth contact portions 352e and 352f in the vertical direction, and may have a shape corresponding to the fifth and sixth contact portions 352e and 352f, respectively. A portion of the third electrode 351c, for example, a portion of the fifth contact portion 352e, may be inserted into the fifth hole 313e, and a portion of the third electrode 351c, for example, a portion of the sixth contact portion 352f, may be inserted and disposed in the sixth hole 313f.

In this case, an area of each of the fifth and sixth contact portions 352e and 352f is disposed below the cover 312, and the remaining area of each of each of the sixth and fifth contact portions 352es and 352f may protrude from the upper surface of the cover 312 to be in contact with the user's skin.

In addition, the seventh hole 313g may be disposed in a region that overlapping the seventh contact portion 352g in the vertical direction and may have a shape corresponding to the seventh contact portion 350g. A portion of the fourth electrode 351d, for example, a portion of the seventh contact portion 352g, may be inserted and disposed in the seventh hole 313g.

In this case, an area of the seventh contact portion 352g is disposed below the cover 312, and the remaining area of the seventh contact portion 352g may protrude from the upper surface of the cover 312 to be in contact with the user's skin.

The skin care device 1 of the embodiment configured as described above, the first to sixth connection portions 353a, 353b, 353c, 353d, 353e, and 353f are formed as cosmetic movement passages in the first, second, and third electrodes 351a, 351b, and 351c disposed in the head portion 3 among the plurality of first, second, third, and fourth electrodes 351a and 351b and 351c and 351d, so that cosmetics may be evenly spread to all electrodes 351a. 351b. 351c, 351d along the movement passages during use, and a connection portion is not formed as a movement passage in the fourth electrode 351d disposed at the outermost edge of the head portion 3, so that the cosmetics are evenly spread only inside the head portion during use.

In addition, the skin care device 1 of the embodiment applies different frequencies to the plurality of first, second, third, and fourth electrodes 351a, 351b, 351c, and 351d alternately at a predetermined period, so that high-frequency thermal energy may be evenly transmitted from an upper dermal layer to a lower dermal layer in the first RF mode, and muscle pain may be eliminated and muscles may be activated due to a low-frequency massage function in the EMS (Electrical Muscle Stimulation) mode.

The skin care device 1 of the present invention may operate in an acceleration mode, the first RF mode, the first EMS mode, and the RF+EMS mode by applying a current of a selected frequency to at least two selected electrodes among the plurality of first, second, third, and fourth electrodes 351a, 351b, 351c, and 351d, which will be described with reference to Table 1 below. Here, the acceleration mode may be included in the first RF mode. In this embodiment, it should be noted that the term “acceleration” is used as a concept of rapidly raising the temperature of the skin to a certain level at the same time as using a skin care device. Under this concept, the acceleration mode refers to a kind of acceleration (boosting) section in which a current of a frequency (first frequency to be described later; 2.2 Mhz) that may be heated the fastest for a certain period of time with a duty of 100% is applied before alternating the frequency of the basic operation in order to operate the skin care device 1, quickly relieve the coldness felt when the skin is in contact with metal and cosmetics when used for the first time, and provide the user's skin with a quick feeling of heat and heat transfer to the deep part.

TABLE 1

| | | 1Electrode | 2Electrode | 3Electrode | 4Electrode |
|---|---|---|---|---|---|
| Acceleration | 2.2 MHz | | | RF(−) | RF(+) |
| Mode | (Acceleration Level 1: 5 s, | | | | |
| | Acceleration Level 2: 10 s, | | | | |
| | Acceleration Level 3: 15 s) | | | | |
| RF | Step 1 | RF(−) | RF(+) | | |
| Mode | (RF: 1.8 MHz <−> 1.0 MHz) | | | | |
| | Step 2 | | | RF(−) | RF(+) |
| | (RF: 2.2 MHz <−> 1.4 Mhz) | | | | |
| EMS | Step 3 | | | EMS (−) | EMS(+) |
| Mode | (EMS: 50 Hz) | | | | |
| | Step 4 | EMS (−) | | | EMS(+) |
| | (EMS: 50 Hz) | | | | |
| | Step 5 (EMS: 50 Hz) | EMS (−) | EMS(+) | | |
| RF + EMS | Step 6 | RF(−) | RF(+) | EMS (−) | EMS(+) |
| Mode | (RF: 2.2 MHz <−> 1.8 MHz), | | | | |
| | (EMS: 50 Hz) | | | | |
| | Step 7 | EMS (−) | RF(+) | RF(−) | EMS(+) |
| | (RF: 1.8 MHz <−> 1.4 MHz), | | | | |
| | (EMS: 50 Hz) | | | | |
| | Step 8 (RF: 1.4 MHz <−> 1.0 MHz), | EMS (−) | EMS(+) | RF(−) | RF(+) |
| | (EMS: 50 Hz) | | | | |

High-frequency waves have different depths of penetration into the skin depending on the wavelength. Therefore, the skin care device 1 of the embodiment may set four frequencies to be used for the four first, second, third, and fourth electrodes 351a, 351b, 351c, and 351d in the range of 1 MHz to 3 MHz (high frequency) so that the deep heat may be deeply covered from the upper dermal layer to the lower dermal layer in the first RF mode. The four frequencies may be set to the first frequency, a second frequency, a third frequency, and a fourth frequency in an order from longest to shortest frequency. For example, the first frequency may be 2.2 MHz, the second frequency may be 1.8 MHz, the third frequency may be 1.4 MHz, and the fourth frequency may be 1.0 MHz, but the present disclosure is not limited thereto.

The first, second, third, and fourth frequencies may be selectively applied to the acceleration mode for rapid temperature rise and the first RF mode massaged by the head portion 3.

In the acceleration mode, the skin temperature is rapidly increased to a certain level at the same time as the device is used, thereby improving the speed at which deep heat is felt.

The acceleration mode may be operated by the user selecting one of the acceleration level 1, the acceleration level 2, and the acceleration level 3 with the function button 27. The acceleration level 1, the acceleration level 2, and the acceleration level 3 may be set in the order from the short acceleration time to the long acceleration time. For example, the acceleration level 1 may be set to 5 seconds, the acceleration level 2 to 10 seconds, and the acceleration level 3 to 15 seconds.

In the acceleration mode, current may be supplied to the third and fourth electrodes 351c and 351d disposed outside the electrode portion 35 among the first, second, third, and fourth electrodes 352a, 351b, 351c, and 351d to accelerate the head portion 3. In this case, the current of the first frequency (2.2 MHz), which has the largest frequency, may be supplied at a duty of 100% to the third and fourth electrodes 351c and 351d to achieve a faster temperature increase rate compared to the target.

In the above, a negative current may be applied to one of the third and fourth electrodes 351c and 351d to which the current of the first frequency 2.2 MHz is applied, and a positive current may be applied on the other electrode. For example, the negative current may be applied to the third electrode 351c, and the positive current may be applied the fourth electrode 351d.

The first RF mode may be automatically executed for a certain period of time from when the acceleration mode is completed, and may be automatically stopped when the certain period of time elapses.

The first RF mode may be divided into step 1 and step 2 in such a manner that when two of the first, second, third, and fourth electrodes 351a, 351b, 351c, and 351d are alternately applied with two of the frequency currents of the first, two, three, and four frequencies at a predetermined period, no current is supplied to the other two electrodes.

For example, in step 1, a current of the second frequency (1.8 MHz) and a current of the fourth frequency (1.0 MHz) may be alternately applied to the first electrode 351a and the second electrode 351b at a period of less than 1 second, and in step 2, the current of the first frequency (2.2 MHz) and a current of the third frequency (1.4 MHz) may alternatively be applied to the third electrode 351c and the fourth electrode 351d at a period of less than 1 second. The alternating period may be between 1 and 999 milliseconds.

Step 2 is not executed when the first RF mode is executed in step 1, and step 1 is not executed when the first RF mode is executed in step 2. Steps 1 and 2 may be alternately executed for a set time, and when the set time is reached, the first RF mode may be automatically stopped. The execution process of steps 1 and 2 will be described in detail below.

In step 1, the current of the second frequency (1.8 MHz) and the current of the fourth frequency (1.0 MHz) may be alternately supplied to the first electrode 351a and the second electrode 351b several times to several tens of times during a first set time.

In the above, a negative current may be applied to one of the two first and second electrodes 351a and 351b to which the current of the second frequency and the current of the fourth frequency is applied, and a positive current may be applied on the other electrode. For example, a negative current may be applied to the first electrode 351a and a positive current may be applied the second electrode 351b.

In step 2, the current of the first frequency (2.2 MHz) and the current of the third frequency (1.4 MHz) may be alternately supplied to the third electrode 351c and the fourth electrode 351d several times to several tens of times during the first set time.

In the above, a negative current may be applied to one of the two third and fourth electrodes 351c and 351d to which the current of the first frequency and the current of the third frequency is applied, and a positive current may be applied on the other electrode. For example, a negative current may be applied to the third electrode 351c, and a positive current may be applied the fourth electrode 351d.

Between step 1 and step 2, a current of 0 MHz frequency is supplied (current off state) to each of the first, second, third, and fourth electrodes 351a, 351b, 351c, and 351d during a second set time.

In the above, the first set time and the second set time may vary depending on the acceleration level selected in the acceleration mode.

For example, in the acceleration mode, when the acceleration level 1 in which the current of the first frequency (2.2 MHz) is supplied to the third and fourth electrodes 351c and 351d for 5 seconds is selected, the first set time during which step 1 or step 2 is executed may be 30 milliseconds, and the second set time during which step 1 and step 2 are not executed may be 20 milliseconds, so that an acceleration temperature corresponding to the acceleration level 1 may be maintained.

In this case, the current of the second frequency (1.8 MHz) and the current of the fourth frequency (1.0 MHz) applied to the first electrode 351a and the second electrode 351b, respectively, and the current of the first frequency (2.2 MHz) and the current of the third frequency (1.4 MHz), respectively, applied to the third electrode 351c and the fourth electrode 351d, respectively, may be supplied with a duty of 60% so as to maintain the acceleration temperature corresponding to the acceleration level 1, and the present invention is not limited thereto.

In addition, in the acceleration mode, when the acceleration level 2 in which the current of the first frequency (2.2 MHz) is supplied to the third and fourth electrodes 351c and 351d for 10 seconds is selected, the first set time during which step 1 or step 2 is executed may be 40 milliseconds, and the second set time during which step 1 and step 2 are not executed may be 10 milliseconds, so that an acceleration temperature corresponding to the acceleration level 2 may be maintained.

In this case, the current of the second frequency (1.8 MHz) and the current of the fourth frequency (1.0 MHz) applied to the first electrode 351a and the second electrode 351b, respectively, and the current of the first frequency (2.2 MHz) and the current of the third frequency (1.4 MHz), respectively, applied to the third electrode 351c and the fourth electrode 351d, respectively, may be supplied at a duty of 80% so as to maintain the acceleration temperature corresponding to the acceleration level 2, and the present invention is not limited thereto.

In addition, when the acceleration level 3 is selected in which the current of the first frequency (2.2 MHz) is supplied to the third and fourth electrodes 351c and 351d for 15 seconds in the acceleration mode, the first set time during which step 1 or step 2 is executed may be 45 milliseconds, and the second set time during which step 1 and step 2 are not executed may be 5 milliseconds, so that an acceleration temperature corresponding to the acceleration level 3 may be maintained.

In this case, the current of the second frequency (1.8 MHz) and the current of the fourth frequency (1.0 MHz) applied to the first electrode 351a and the second electrode 351b, respectively, and the current of the first frequency (2.2 MHz) and the current of the third frequency (1.4 MHz) applied to the third electrode 351c and the fourth electrode 351d, respectively, may be supplied with a duty of 90% so as to maintain the acceleration temperature corresponding to the acceleration level 3, and the present invention is not limited thereto.

In the, the first RF mode of the skin care device 1 may select one of the acceleration level 1, the acceleration level 2, and the acceleration level 3, and the first and second electrodes 351a and 351b of step 1 and the third and fourth electrodes 351c and 351d of step 2, which are executed in the first RF mode after the acceleration mode, may be repeatedly executed in a certain order. The first RF mode may be repeatedly executed for a predetermined time in the order of step 1->step 2->step 1->step 2. In this case, the total execution time of the first RF mode may be set to about 5 minutes for the safety of the user, but is not limited thereto.

In order to eliminate muscle pain and activate muscles, the skin care device 1 of the embodiment may execute the first EMS mode by setting at least one fifth frequency to be used for the four first, second, third, and fourth electrodes 351a, 351b, 351c, and 351d, for example, setting at least one of the fifth frequency within a range of 150 Hz (low frequency) or less, and setting various execution times.

The first EMS mode may be automatically executed for a certain period of time from the time when the acceleration mode is completed, and the first EMS mode may be automatically stopped after the certain period of time lapses. In addition, the first EMS mode may be executed independently regardless of the acceleration mode.

The first EMS mode may be divided into step 3, step 4, and step 5 in such a manner that when a current of the fifth frequency is applied to two of the four first, second, third, and fourth electrodes 351a, 351b, 351c, and 351d at a predetermined period, no current is supplied to the other two electrodes. The skin care device 1 of the embodiment is described by setting the fifth frequency to 50 Hz, but is not limited thereto.

For example, in step 3 of the first EMS mode, the current of the fifth frequency (50 Hz) may be applied to each of the third electrode 351c and the fourth electrode 351d, and in this case, no current is applied to the first electrode 351a and the second electrode 351b. In step 4 of the first EMS mode, the current of the fifth frequency (50 Hz) may be applied to each of the first electrode 351a and the fourth electrode 351d, and in this case, no current is applied to the second electrode 351b and the third electrode 351c. In step 5 of the first EMS mode, the current of the fifth frequency (50 Hz) may be applied to each of the first electrode 351a and the second electrode 351b, and in this case, no current is applied to the third electrode 351c and the fourth electrode 351d.

The fifth frequency applied to each of step 3, step 4, and step 5 in the first EMS mode may be the same, but is not limited thereto.

The first EMS mode may be executed by varying the stimulation time in each of step 3, step 4, and step 5 to which the fifth frequency is applied. The first EMS mode may be operated by the user selecting one of a stimulation level 1, a stimulation level 2, and a stimulus level 3 with the function button 27. The stimulation level 1, the stimulation level 2, and the stimulation level 3 are classified according to the stimulation time, and the stimulation time of the stimulation level 1 is the shortest and that of the stimulation level 3 is the longest. For example, the stimulation level 1 may be set to 10 microseconds, the stimulation level 2 may be set to 12 microseconds, and the stimulation level 3 may be set to 14 microseconds, but are not limited thereto.

In addition, in the first EMS mode, a current with a frequency of 0 Hz is supplied (current off state) to each of the first, second, third, and fourth electrodes 351a, 351b, 351c, and 351d for a certain period of time, for example, 20 microseconds between each of step 3, step 4, and step 5.

A negative current may be applied to one of the two third and fourth electrodes 351c and 351d, the two first and fourth electrodes 352a and 351d or the two first and second electrodes 351a and 351b to which the current of the fifth frequency is applied, and a positive current may be applied on the other electrode. For example, a negative current may be applied to the former of the two electrodes and a positive current to the latter.

Step 4 and step 5 are not executed when the first EMS mode is executed in step 3, step 3 and step 5 are not executed when the first EMS Mode is executed in step 4, and step 3 and step 4 are not executed when the first EPS mode is executed in step 5.

As described above, in the first EMS mode of the skin care device 1, any one of the stimulation level 1, the stimulation level 2, and the stimulation level 3 may be selected, and the third and fourth electrodes 351c and 351d of step 3, the first and fourth electrodes 352a and 351d of step 4, and the first and second electrodes 351a and 351b of step 5, which are executed in the first EPS mode, may be repeatedly executed in a certain order. The first EMS mode of the skin care device 1 may be repeatedly executed for a predetermined time in the order of step 3->step 4->step 5->step 4->step 3->step 4->step 5->step 4. In this case, the total execution time of the first EMS mode may be set to about 5 minutes for the safety of the user, but is not limited thereto.

The RF+EMS mode of the skin care device 1 may be a mode in which a second RF mode and a second EMS mode are executed in combination. The RF+EMS mode may be implemented by executing the second RF mode and the second EMS mode simultaneously, adding the second EMS Mode during execution of the second RF mode, or additionally executing the second RF Mode during execution of the second EMS mode.

The RF+EMS mode of the skin care device 1 of the embodiment may be divided into step 6, step 7, and step 8 in such a manner that the second RF mode is executed on two of the four first, second, third, and fourth electrodes 351a, 351b, 351c, and 351d, and the second EMS mode is executed on the other two electrodes.

In step 6 of the RF+EMS mode, the first electrode 351a and the second electrode 351b may be executed in the second RF mode, and the third electrode 351c and the fourth electrode 351d may be executed in the second EMS mode.

The current of the first frequency (2.2 MHz) and the current of the second frequency (1.8 MHz) may be alternately applied to each of the first electrode 351a and the second electrode 351b that are executed in the second RF mode at a period of less than one second.

Each of the third electrode 351c and the fourth electrode 351d, which are executed in the second EMS mode, selects the stimulation level 1, the stimulation level 2, or the stimulation level 3, so that the current of the fifth frequency (50 Hz) may be applied for a time of 10 microseconds, 12 microseconds, or 14 microseconds.

In step 6 of the RF+EMS mode, the second RF mode is different from step 1 of the first RF mode only in the frequency applied to the first electrode 351a and the second electrode 351b being executed, and the remaining conditions may be the same or similar, and thus, in order to avoid duplicate descriptions, detailed descriptions will be omitted herein.

In addition, in step 6 of the RF+EMS mode, the second EMS mode may have the same electrodes and the same or similar conditions as in step 3 of the first EMS mode, and thus, in order to avoid duplicate description, detailed description will be omitted herein.

In step 7 of the RF+EMS mode, the second electrode 351b and the third electrode 351c may be executed in the second RF mode, and the first electrode 351a and the fourth electrode 351d may be executed in the second EMS mode.

The current of the second frequency (1.8 MHz) and the third frequency (1.4 MHz) may be alternately applied to each of the second electrode 351b and the third electrode 351c that are executed in the second RF mode at a period of less than 1 second.

Each of the first electrode 351a and the fourth electrode 351d, which are executed in the second EMS mode, selects the stimulation level 1, the stimulation level 2, or the stimulation level 3, so that the current of the fifth frequency (50 Hz) may be applied for a time of 10 microseconds, 12 microseconds, or 14 microseconds.

In step 7 of the RF+EMS mode, the second RF mode is different from step 1 of the first RF mode only in the frequency applied to the second electrode 351b being executed, and the remaining conditions may be the same or similar, and the second RF mode and step 2 of the first RF Mode are different from step 2 of the second RF mode in only the frequency applied to the third electrode 351c being executed, and thus, in order to avoid duplicate descriptions, detailed descriptions will be omitted herein.

In addition, in step 7 of the RF+EMS mode, the second EMS mode may have the same electrodes and the same or similar conditions as in step 4 of the first EMS mode, and thus, in order to avoid duplicate descriptions, detailed descriptions will be omitted herein.

In step 8 of the RF+EMS mode, the third electrode 351c and the fourth electrode 351d may be executed in the second RF mode, and the first electrode 351a and the second electrode 351b may be executed in the second EMS mode.

The current of the third frequency (1.4 MHz) and the current of the fourth frequency (1.0 MHz) may be alternately applied to each of the third electrode 351c and the fourth electrode 351d that are executed in the second RF mode at a period of less than one second.

Each of the first electrode 351a and the second electrode 351b, which are executed in the second EMS mode, selects the stimulation level 1, the stimulation level 2, or the stimulation level 3, so that the current of the fifth frequency (50 Hz) may be applied for a time of 10 microseconds, 12 microseconds, or 14 microseconds.

In step 8 of the RF+EMS mode, the second RF mode is different from step 2 of the first RF mode only in the frequency applied to the third electrode 351c and the fourth electrode 351d, and the remaining conditions may be the same or similar, and accordingly, detailed descriptions will be omitted here to avoid duplicate descriptions.

In addition, in step 8 of the RF+EMS mode, the second EMS mode may have the same electrodes and the same or similar conditions as in step 5 of the first EMS mode, and thus, in order to avoid duplicate descriptions, detailed descriptions will be omitted herein.

As described above, the second RF mode of each of step 6, step 7, and step 8 in the RF+EMS mode of the skin care device 1 may be executed to correspond to step 1 and step 2 of the first RF mode. The second EMS mode of each of step 6, step 7, and step 8 in the RF+EMS mode of the skin care device 1 may be executed to correspond to step 3, step 4, and step 5 of the first EMS mode.

In the RF+EMS mode of the skin care device 1, step 6, step 7, and step 8 may be performed in combination with the execution process of step land step 2 of the first RF mode and the execution process of steps 3, 4, and 5 of the first EMS mode. However, the order of execution may be different.

That is, in the RF+EMS mode, one of the acceleration level 1, the acceleration level 2, and the acceleration level 3 may be selected for the RF mode, and the first and second electrodes 351a and 351b of step 6, the second and third electrodes 351b and 351c of step 7, and the third and fourth electrodes 351c and 351d of step 8, which are executed in the second RF mode after the acceleration mode, may be repeatedly executed in a certain order. In the RF+EMS mode, the second RF mode may be repeatedly executed for a certain period of time in the order of step 6->step 7->step 8->step 7->step 6->step 7->step 8->step 7.

In addition, the third and fourth electrodes 351c and 351d of step 6, the first and fourth electrodes 352a and 351d of step 7, and the first and second electrodes 351a and 351b of step 8, which are executed from the RF+EMS mode to the second EMS mode, may be repeatedly executed in a certain order. In the RF+EMS mode, the second EMS mode may be repeatedly executed for a certain period of time in the order of step 6->step 7->step 8->step 7->step 6->step 7->step 8->step 7.

As a result of testing the human body in the RF mode of the skin care device 1 of the embodiment, the following results were obtained.

### 1. Skin Wrinkle Analysis

\textcircled{1} Wrinkles around the eyes; Compared to before using the product, the overall size, depth, and maximum depth parameters decreased significantly immediately after using the product and after 4 weeks of using the product (p<0.05), and the reduction rate was 8.87% to 14.52%.

\textcircled{2} Nasolabial folds; Compared to before using the product, the overall size and depth parameters decreased significantly immediately after using the product and 4 weeks after using the product, and the width parameter decreased significantly after 4 weeks of using the product (p<0.05), and the reduction rate was 3.43% to 10.08%.

\textcircled{3} Forehead wrinkles; Compared to before using the product, the overall size and depth parameters decreased significantly immediately after using the product and after 4 weeks (p<0.05), and the reduction rate was 2.14% to 10.08%.

\textcircled{4} Glabellar wrinkles; Compared to before using the product, the overall size, depth and maximum depth parameters decreased significantly immediately and 4 weeks after using the product, and the width parameter decreased significantly after 4 weeks of using the product (p<0.05), and the reduction rate of 1.85% to 11.78%.

### 2. Skin Pore Analysis

When compared to before using the product, the volume, count, and density parameters decreased significantly at 2 and 4 weeks after using the product (p<0.05), and the reduction rate was 6.79% to 14.66%.

### 3. Skin Brightness Analysis

When compared to before using the product, the hyper concentration parameter of the pigmented area significantly decreased 2 and 4 weeks after using the product, and the affected area parameter decreased significantly after 4 weeks of using the product (p<0.05). The reduction rates were 7.44% and 11.16% for the hyper concentration parameter and 14.07% for the affected area parameter, respectively.

### 4. Analysis of Elastic Recovery of Skin Pressure Marks

When compared to before using the product, the elastic recovery of skin pressure marks significantly increased after 2 and 4 weeks of using the product (p<0.05), and the reduction rates were 12.78% and 21.82%, respectively.

### 5. Skin Moisture (Density) Analysis

When compared to before using the product, skin moisture (density) significantly increased immediately after and 4 weeks after using the product (p<0.05), and the increase rates were 226.79% and 12.07%, respectively.

### 6. Skin Density Analysis

When compared to before using the product, skin density significantly increased 2 and 4 weeks after using the temporary product (p<0.05), and the increase rates were 8.60% and 17.90%, respectively.

### 7. Skin Lifting Analysis

When compared to before using the product, the volume of the double chin area decreased significantly immediately and 4 weeks after using the product (p<0.05), and the reduction rates were 10.14% and 21.74%, respectively.

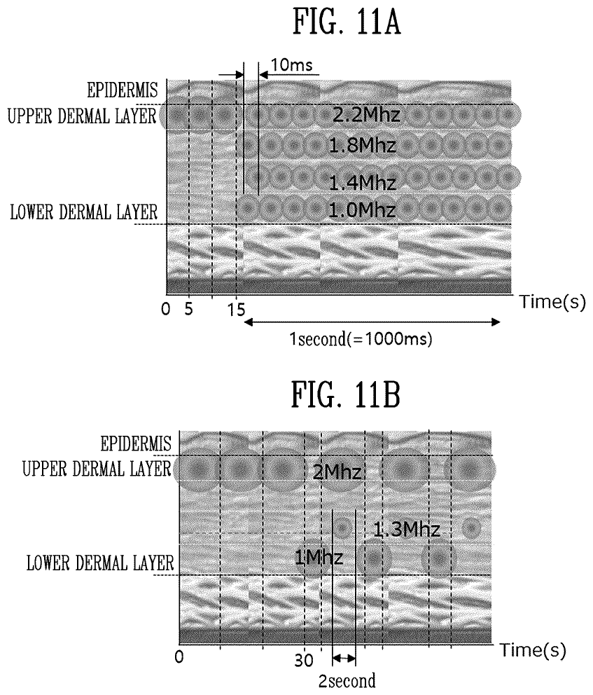

In addition, as it may be seen from FIG. 11(a) which illustrates that thermal energy is transferred to the dermis layer by the skin care device 1 of the present invention, and FIG. 11(b) which illustrates thermal energy is transferred to the dermis layer by the skin care device of the comparative invention, the present invention was excellent in terms of thermal energy transfer efficiency, such as that thermal energy is evenly transferred from the upper and lower dermal layers, but thermal energy is not transferred to the lower dermal layer when thermal energy is transmitted to the upper dermal layer in the thermal energy transmission in the skin care device of the comparative invention. Here, the skin care device of the comparative invention uses fixed RF frequency electrodes and changes the frequency in time units of 2 seconds or more to simultaneously transmit energy to the epidermis and lower dermal layers, and is one of the products currently on the market.

The features, structures, effects, etc. described in the embodiments above are included in at least one embodiment of the present invention and are not necessarily limited to only one embodiment. Furthermore, the features, structures, effects, etc. illustrated in each embodiment can be combined or modified and implemented in other embodiments by a person with ordinary knowledge in the field to which the embodiments belong. Therefore, contents related to such combinations and modifications should be construed as being included in the scope of the present invention.

In addition, although the above description has been made focusing on the examples, this is only an example and does not limit the present invention, and those skilled in the art will understand the above examples without departing from the essential characteristics of the present embodiment. You will be able to see that various modifications and applications are possible. For example, each component specifically shown in the examples can be modified and implemented. And these variations and differences in application should be construed as being included in the scope of the present invention as defined in the appended claims.

The invention claimed is:

1. A skin care device comprising:
a body including a first terminal; and
a head portion connected to the body and detachable from the body,
wherein the head portion includes:
a housing including an accommodation space therein;
a substrate disposed in the housing;
an electrode portion disposed in the housing and disposed on the substrate; and
a second terminal located below the substrate and electrically connected to the first terminal of the body;
wherein the electrode portion includes:
a first electrode disposed at a center of the head portion;
a second electrode surrounding a periphery of the first electrode and disposed alternately at equal intervals;
a third electrode surrounding a periphery of the second electrode and disposed alternately at equal intervals; and
a fourth electrode surrounding a periphery of the third electrode and disposed alternately at equal intervals,
wherein the first, second, third and fourth electrodes operate in a first RF mode, and
wherein the first RF mode is applied to two of the first, second, third, and fourth electrodes at different frequencies alternately at a predetermined period, so that high-frequency thermal energy is evenly transmitted from an upper dermal layer to a lower dermal layer.

2. The skin care device according to claim 1, wherein the first RF mode sets frequencies to be used for the first, second, third, and fourth electrodes to four frequencies in a range of 1 MHz to 3 MHz (high-frequency), and
wherein the four frequencies are set to a first frequency, a second frequency, a third frequency, and a fourth frequency in an order from longest to shortest frequency.

3. The skin care device according to claim 2,
wherein the first frequency is set to 2.2 MHz,
wherein the second frequency is set to 1.8 MHz,
wherein the third frequency is set to 1.4 MHz, and
wherein the fourth frequency is set to 1.0 MHz.

4. The skin care device according to claim 2, further comprising an acceleration mode for rapid thermal transfer to a skin before operating the first RF mode,
wherein one of acceleration level 1, acceleration level 2, and acceleration level 3 which are set in an order from longest to shortest time is selected and operated in the acceleration mode,
wherein the third and fourth electrodes are supplied with a current of the first frequency at a duty of 100%,
wherein a negative current is applied to one of the third and fourth electrodes and a positive current is applied to an other electrode.

5. The skin care device according to claim 4, wherein the first RF mode is executed for a certain period of time from when the acceleration mode is completed and is automatically stopped after the certain period of time lapses, and
wherein the first RF mode is divided into step 1 and step 2 which are separately executed in a manner of which when two of the first, second, third, and fourth electrodes are alternately applied with two of the frequency currents of the first, two, three, and four frequencies at a predetermined period, no current is supplied to the other two electrodes.

6. The skin care device according to claim 5,
wherein the first RF mode is configured to:
apply the currents of the second frequency and the fourth frequency alternately to each of the first and second electrodes several times to several tens of times at a period of less than 1 second during a first set time in the step 1; and
apply the currents of the first frequency and the third frequency alternately to the third and fourth electrodes several times to several tens of times at the period of less than 1 second during the first set time in the step 2, and
wherein a negative current is applied to one of the two first and second electrodes or the two third and fourth electrodes, and a positive current is applied to an other electrode, and
wherein the step 1 and the step 2 are alternately executed with a gap of a second set time during which no current is supplied to each of the first, second, third, and fourth electrodes.

7. The skin care device according to claim 6, wherein the first set time becomes longer from the acceleration level 1 to the acceleration level 3, and
wherein the second set time becomes relatively shorter from the acceleration level 1 to the acceleration level 3.

8. The skin care device according to claim 5, wherein the first RF mode is repeatedly executed for the certain period of time in an order of the step 1->the step 2->the step 1→the step 2 after the acceleration mode.

9. The skin care device according to claim 1,
wherein the first, second, third, and fourth electrodes operate in a first EMS mode instead of the first RF mode,
wherein the first EMS mode is divided into step 3, step 4, and step 5 which are separately executed in a manner of which when a current of a fifth frequency is applied to two of the first, second, third, and fourth electrodes at a predetermined period, no current is supplied to other two electrodes,
wherein the first EMS mode is configured to:
apply the current of the fifth frequency to each of the third and fourth electrodes in the step 3;
apply the current of the fifth frequency to each of the first and fourth electrodes in the step 4;
apply the current of the fifth frequency to each of the first and second electrodes in the step 5, and
wherein a negative current is applied to one of the two third and fourth electrodes, the two first and fourth electrodes, or the two first and second electrodes, and a positive current is applied to an other electrode.

10. The skin care device according to claim 9, wherein, in the first EMS mode, one of stimulation level 1, stimulation level 2, and stimulation level 3 which are set in an order from shortest to longest is selected and executed so that a stimulation time in each of the step 3, the step 4, and the step 5 is different, and
wherein between each of the step 3, the step 4, and the step 5, no current is supplied to each of the first, second, third, and fourth electrodes for a certain period of time.

11. The skin care device according to claim 9, wherein the first EMS mode is repeatedly executed for a certain period of time in an order of the step 3->step 4->step 5->step 4->step 3->step 4->step 5->step 4.

12. The skin care device according to claim 1, wherein the first, second, third, and fourth electrodes operate in a RF+EMS mode instead of the first RF mode,
wherein the RF+EMS mode is divided into step 6, step 7, and step 8 which are separately executed in a manner of which when the second RF mode is operated in two of the first, second, third, and fourth electrodes, and the second EMS mode is operated in other two electrodes.

13. The skin care device according to claim 12,
wherein the first and second electrodes are executed in the second RF mode and the third and fourth electrodes are executed in the second EMS mode in the step 6,
wherein the second and third electrodes are executed in the second RF mode and the first and fourth electrodes are executed in the second EMS mode in the step 7, and
wherein the third and fourth electrodes are executed in the second RF mode and the first and second electrodes are executed in the second EMS mode in the step 8.

14. The skin care device according to claim 13, wherein the second RF mode sets frequencies to be used for the first, second, third, and fourth electrodes to four frequencies in a range of 1 MHz to 3 MHz (high frequency),
wherein the four frequencies are set to a first frequency, a second frequency, a third frequency, and a fourth frequency in an order from longest to shortest frequency, and
wherein the second EMS mode sets frequencies to be used for the first, second, third, and fourth electrodes to five frequencies in a low frequency range.

15. The skin care device according to claim 14, further comprising an acceleration mode for rapid thermal transfer to a skin before operating the second RF mode,
wherein one of acceleration level 1, acceleration level 2, and acceleration level 3 which are set in an order from longest to shortest time is selected and operated in the acceleration mode,
wherein the third and fourth electrodes are supplied with a current of the first frequency at a duty of 100%,
wherein a negative current is applied to one of the third and fourth electrodes and a positive current is applied to an other electrode.

16. The skin care device according to claim 15,
wherein second RF mode is configured to:
apply the currents of the first frequency and the second frequency alternately to each of the first and second electrodes several times to several tens of times at a period of less than 1 second during a first set time in the step 6;
apply the currents of the second frequency and the third frequency alternately to each of the second and third electrodes several times to several tens of times at the period of less than 1 second during the first set time in the step 7; and
apply the currents of the third frequency and the fourth frequency alternately to the third and fourth electrodes several times to several tens of times at the period of less than 1 second during the first set time in the step 8,
wherein a negative current is applied to one electrode of the two first and second electrodes, the two second and third electrodes, or the two third and fourth electrodes, and a positive current is applied to an other electrode,
wherein the step 6, the step 7, and the step 8 are alternately executed with a gap of a second set time during which no current is supplied to each of the first, second, third, and fourth electrodes,
wherein the first set time becomes longer from the acceleration level 1 to the acceleration level 3,
wherein the second set time becomes relatively shorter from the acceleration level 1 to the acceleration level 3,
wherein the second EMS mode is configured to:
apply a current of the fifth frequency to each of the third and fourth electrodes in the step 6;
apply the current of the fifth frequency to each of the first and fourth electrodes in the step 7; and
apply the current of the fifth frequency to each of the first and second electrodes in the step 8, and
wherein a negative current is applied to one of the two third and fourth electrodes, the two first and fourth electrodes, or the two first and second electrodes, and a positive current is applied to an other electrode.

17. The skin care device according to claim 16, wherein, in the second EMS mode, one of stimulation level 1, stimulation level 2, and stimulation level 3 which are set in an order from shortest to longest is selected and executed so that a stimulus time in each of the step 6, the step 7, and the step 8 is different, and
wherein between each of the step 6, the step 7, and the step 8, no current is supplied to each of the first, second, third, and fourth electrodes for a certain period of time.

18. The skin care device according to claim 12, wherein the RF+EMS mode is repeatedly executed for a certain period of time in an order of the step 6->step 7->step 8->step 7->step 6->step 7->step 8->step 7.
